(19) United States
(12) Patent Application Publication
(10) Pub. No.: US 2025/0143566 A1
(43) Pub. Date: May 8, 2025

(54) PHASE-BASED OPTORETINOGRAPHY USING TISSUE VELOCITY

(71) Applicant: THE REGENTS OF THE UNIVERSITY OF CALIFORNIA, Oakland, CA (US)

(72) Inventors: Ravi S. Jonnal, Davis, CA (US); Robert J. Zawadzki, Davis, CA (US); Kari V. Vienola, Davis, CA (US)

(73) Assignee: THE REGENTS OF THE UNIVERSITY OF CALIFORNIA, Oakland, CA (US)

(21) Appl. No.: 18/940,045

(22) Filed: Nov. 7, 2024

Related U.S. Application Data
(63) Continuation of application No. PCT/US2023/021827, filed on May 11, 2023.
(60) Provisional application No. 63/340,804, filed on May 11, 2022.

(51) Int. Cl. A61B 3/10

(52) U.S. Cl. CPC A61B 3/102

(57) ABSTRACT

Phase-based optoretinography (ORG) is performed using tissue velocity obtained from a modified optical coherence tomography (OCT) system. A swept-source generated OCT A-scan is used to assemble a 2-dimensional profile of the retinal tissue (B-scan) by laterally scanning the imaging beam. These B-scans can be taken at multiple time-points and assembled into a M-scan, which displays motion of a particular cross-section of retinal tissue (after correction for bulk motion). The method captures the changes between each timestep of the M-scan to deduce tissue velocities of the COST, ROST, and IS/OS, among others. An initial contraction of outer segments is detected in outer segments, followed by an elongation shortly thereafter. The tissue velocity measurements can be used to infer retinal dysfunction without the need for costly and computationally expensive scans using adaptive optics.

### CROSS-REFERENCE TO RELATED APPLICATIONS

This application claims priority to, and is a 35 U.S.C. § 111 (a) continuation of, PCT international application number PCT/US2023/021827 filed on May 11, 2023, incorporated herein by reference in its entirety, which claims priority to, and the benefit of, U.S. provisional patent application Ser. No. 63/340,804 filed on May 11, 2022, incorporated herein by reference in its entirety. Priority is claimed to each of the foregoing applications.

The above-referenced PCT international application was published as PCT International Publication No. WO 2023/220235 A1 on Nov. 6, 2023, which publication is incorporated herein by reference in its entirety.

### STATEMENT REGARDING FEDERALLY SPONSORED RESEARCH OR DEVELOPMENT

This invention was made with Government support under Grant Nos. RO1 EY026556, RO1 EY033532, and ROO EY026068, awarded by the National Institutes of Health (NIH). The Government has certain rights in the invention.

### NOTICE OF MATERIAL SUBJECT TO COPYRIGHT PROTECTION

A portion of the material in this patent document may be subject to copyright protection under the copyright laws of the United States and of other countries. The owner of the copyright rights has no objection to the facsimile reproduction by anyone of the patent document or the patent disclosure, as it appears in the United States Patent and Trademark Office publicly available file or records, but otherwise reserves all copyright rights whatsoever. The copyright owner does not hereby waive any of its rights to have this patent document maintained in secrecy, including without limitation its rights pursuant to 37 C.F.R. § 1.14.

### BACKGROUND

### 1. Technical Field

The technology of this disclosure pertains generally to devices and methods for all-optical testing of neural function in the retina, and more particularly to optoretinography (ORG) methods using state-of-the-art optical imaging systems and devices based on optical coherence tomography (OCT).

### 2. Background Discussion

Visual information is gathered in the retina by photoreceptors as they absorb photons and convert their energy into membrane potentials in a process known as phototransduction. The resulting signal propagates through several classes of retinal interneurons before being transmitted to the brain via the optic nerve.

A schematic overview of the anatomy of an eye 100 and the location of the retina 102 are shown in FIG. 1A, which also shows the cornea 104, anterior chamber 106, pupil 108, iris 110, lens 112, ciliary body 114, sclera 116, fovea 118, choroid 120, optic nerve head 122, and vitreous 124. Assessment of the visual process and its cellular mechanisms is indispensable for disease assessment in the clinic and studies of the mechanisms of disease and efficacy of therapeutic interventions. To that end, a variety of psychophysical and electrophysiological tests have been used. Some of these, such as eye charts and perimetry, are subjective, in the sense that they require feedback from the patient regarding the visibility of stimuli. Subjective tests can be time-consuming and suffer from spurious sources of variance such as attention and learning effects. Others, like the electroretinogram (ERG), permit objective measurement of stimulus-evoked electrical activity, but are moderately invasive, requiring placement of electrodes on the cornea and face.

In addition to assessing visual function, clinicians need to observe how the retinal structure is affected by disease. Historically, this was done with slit lamp exams and fundus photography, but over the past two decades, optical coherence tomography (OCT) has become a standard of ophthalmic care. With OCT, the laminar structure 126 of the retina 102 can be visualized non-invasively in three dimensions as illustrated in the image 128 shown in FIG. 1B. Even with much progress in the development of tools for both functional and structural assessment, a need exists for an objective, noninvasive assay of retinal function, ideally capable of simultaneously observing the structure of the retina.

As an interferometric imaging modality, OCT records the amplitude and phase of light reflected by the retina. The familiar OCT image is created using only the signal amplitude, while the phase is disregarded. However, the phase of the signal contains information about the position of scattering objects in the scene, and is thus sensitive to tissue movement, even if it is much smaller than the optical resolution of the imaging system. Sensitivity to the movement of a structure depends on its reflectivity: for the brightest features in the ophthalmic OCT image, movements as small as a few nanometers may be measured. This level of sensitivity requires computational methods to correct for the confounding phase shifts caused by eye motion.

Because neurons are known to swell and shrink during signaling, phase-sensitive OCT offers the ability to detect whether these cells are responding to stimuli. Early efforts to observe these stimulus-evoked responses in human cone photoreceptors used adaptive optics (AO), which permitted resolution and tracking of single cells, in conjunction with common path interferometry. Since then, interest in this area has grown, and these responses have been successfully measured in cones using detection schemes such as full-field swept-source OCT with digital aberration correction (DAC), flying-spot AO-OCT, and line-scanning OCT and AO-OCT. Recently, responses have also been measured in rods, using a multimodal AO-OCT system. These cutting-edge systems all provide measurements of neural responses at the level of single cells, where characteristics of the response may be studied in detail and where disease-related dysfunction manifests earliest.

Published reports of light-evoked phase changes in the photoreceptors have used either (1) adaptive optics (AO)-OCT, or (2) full field (FF) OCT with digital aberration correction (DAC). AO-OCT systems have been used to measure ORG responses in photoreceptors by achieving high volumetric imaging rates (6 Hz+). These rates are achieved by using very fast swept-source lasers (1.6 MHz+ A-scan rates), multiplexing multiple detection channels (equivalent to 1 MHZ+ A-scan rates), or using a line-scanning approach with a very advanced 10-100 kHz frame rate CMOS camera (5+ MHz effective A-scan rates). FF-OCT with DAC relies on very fast cameras to achieve volume rates of 100 Hz+ and effective A-scan rates of 10+ MHz. These approaches typically scan a one degree (approximately 300 micrometers) patch of retina, and use post-processing to align the volumes and track single cells over time. The ORG signal is measured in individual cells, and the cellular responses are averaged to reduce noise.

The most commonly reported optoretinographic method has been to localize the two boundaries of the photoreceptor outer segment, i.e., the inner segment-outer segment junction (IS/OS) and the cone or rod outer segment tip (COST or ROST), and monitor the difference between the phase of light returning from the two structures (see FIG. 1C and FIG. 1D). FIG. 1C illustrates the layered structure of the retina consisting of nerve fiber layer 130, the ganglion cell layer 132, the inner plexiform layer 134, the inner nuclear layer 136, the outer plexiform layer 138, the outer nuclear layer 140, the photoreceptors inner segment (IS) 142, the photoreceptors outer segment (OS) 144, and the pigment epithelium 146. FIG. 1D illustrates a cone 148 and a rod 150, as well as the external limiting membrane 152, inner segment 142 (IS), IS/OS junction 154, outer segment (OS) 144, cone outer segment tip (COST) 156, rod outer segment tip (ROST) 158, retinal pigment epithelial cells 146, and Bruch's membrane 160. This method to observe photoreceptor responses in the retina has been termed optoretinography (ORG), and it has been successfully used to classify cones by spectral properties and to detect cone dysfunction in retinitis pigmentosa patients.

It is believed that ORG is the only noninvasive, objective test of neural function in the retina that can simultaneously reveal its structure, making it ideal for ophthalmic care and clinical research. However, the advanced imaging systems used to prove the ORG concept pose some challenges for clinical translation. The systems utilizing AO require expensive components and incur additional costs in personnel due to their optical complexity and need for multiple expert operators. In addition, the data rates of these instruments are in the tens of gigabytes per second which, in conjunction with the required data processing, precludes rapid test results at present. Together, these constraints limit the number of healthy and disease-affected eyes that can be tested, and thus the translational utility of the test.

### BRIEF SUMMARY

This disclosure describes systems and methods for measuring tissue velocities of retinal cellular layers from clinical OCT (Optical Coherence Tomography) scans, such as scans of cone outer segment tips (COST), rod outer segment tips (ROST), and the inner segment/outer segment junction (IS/OS). In one embodiment, a swept-source scan is used to assemble a 2-dimensional profile of the retinal tissue (B-scan). These B-scans can be taken at multiple time-points and assembled into a M-scan, which displays motion of a particular cross-section of retinal tissue (after correction for bulk motion). The method captures the changes between each timestep of the M-scan to deduce tissue velocities of the COST, ROST, and IS/OS, among others. For example, an initial contraction of outer segments is detected in outer segments, followed by an elongation shortly thereafter. These novel tissue velocity measurements can be used to infer retinal dysfunction without the need for costly and computationally expensive scans using adaptive optics.

Note also that the methods described herein assess not just the size of retinal substructures, but specifically measure the tissue velocity of outer segment tips and segment junctions in response to stimulus. The methods provide a data processing pipeline that achieves this tissue velocity measurement when given serial B-scans supplied by common clinical OCT scanners in conjunction with modified scanning protocols, additional phase stabilization, and a subsystem for configuring and delivering visible stimuli. These modifications and assessment of tissue velocity together constitute the novel approach.

Further aspects of the technology described herein will be brought out in the following portions of the specification, wherein the detailed description is for the purpose of fully disclosing preferred embodiments of the technology without placing limitations thereon.

### BRIEF DESCRIPTION OF THE DRAWINGS

The technology described herein will be more fully understood by reference to the following drawings, which are for illustrative purposes only:

FIG. 1A is a schematic diagram of an eye indicating key parts of the eye.

FIG. 1B is a wide field-of-view B-scan image of the human retina expanding from temporal to nasal across the fovea and optic nerve head.

FIG. 1C is an image and drawing showing the different cellular structures of the retina and what they correspond to in a B-scan.

FIG. 1D is a schematic side view of the human photoreceptor cells with details of different structures. For optoretinography, the outer segment (OS) elongation is observed.

FIG. 2 is a schematic diagram of one SS-OCT system layout (not to scale) according to an embodiment of the technology of this disclosure.

FIG. 3 is a diagram of ORG signal processing according to an embodiment of the technology of this disclosure.

FIG. 4 is a flowchart showing an embodiment of ORG signal processing steps according to the technology of this disclosure.

FIG. 5 is a set of images showing phase slopes calculated from the block for each individual pixel in OCT and visualized after bulk motion filtering. The residual velocities in the inner retina are due to blood flow.

FIG. 6 is a perspective view of M-scans that are generated by averaging A-scans within each individual B-scan, creating an averaged depth profile over time.

FIG. 7 shows examples of figures of merit for ORG responses resulting from the technology of this disclosure.

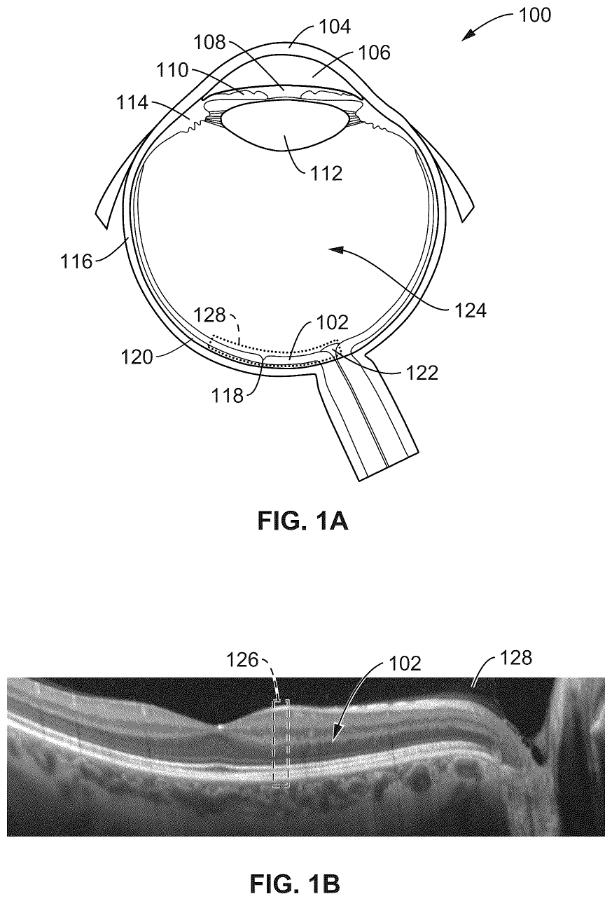

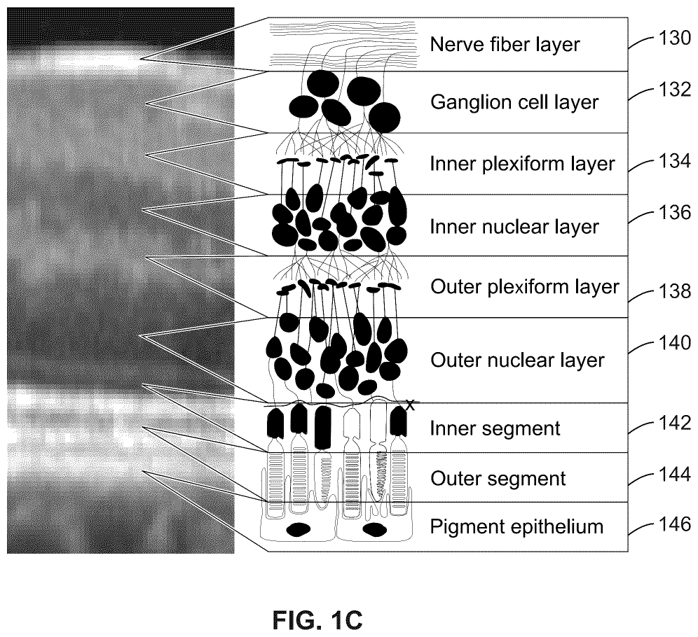

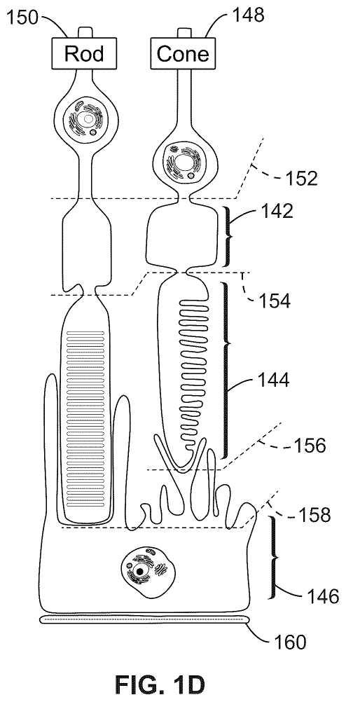

### DETAILED DESCRIPTION

### 1. Introduction

Optoretinography (ORG) is an emerging tool for testing neural function in the retina. Unlike existing methods, it is non-invasive, objective, and provides information about the retinal structure and function at once. As such, it has great potential to transform ophthalmic care and clinical trials of novel therapeutics designed to restore or preserve visual function. Recent efforts have demonstrated the feasibility of the ORG using state-of-the-art optical coherence tomography systems. These methods measure the stimulus-evoked movement of subcellular features in the retina, using the phase of the reflected light to monitor their position.

In contrast, we present an alternative approach that monitors the velocity of these features instead. This conceptual shift has significant implications for the nascent field of optoretinography. By avoiding the need to track specific cells over time, our approach obviates costly and laborious aspects of the position-based approaches, such as adaptive optics, digital aberration correction, real-time tracking, and three-dimensional segmentation and registration.

In the discussion that follows, we present an ORG approach using a custom OCT system very similar to those currently employed in the clinic. However, unlike prior approaches, our approach lacks the ability to resolve and track single cells. Instead, we describe a signal processing pipeline that is designed around the assumption that the exact cells being imaged at any given time may move out of the field of view at other times and depends on the correlated behavior of adjacent cells to extract a meaningful signal. Phase changes in the IS/OS and COST layers are measured within a time window short enough to ignore retinal movement (e.g., <10 ms), and converted into instantaneous, depth-dependent tissue velocities. The resulting series of velocities are related, by integration, to the underlying contraction and expansion of the cone's outer segments. Reconstruction of the tissue position may not be necessary anyway, since previous reports suggest that the derivative of the position signal (i.e., velocity) is an advantageous way to quantify it.

As proof of concept, we used this velocity-based approach to measure the photoreceptor ORG responses in three healthy subjects. The resulting responses were reproducible and exhibited dependence on dose and retinal eccentricity. The position of the relevant structures was reconstructed through numerical integration of velocity.

### 2. Optical Coherence Tomography System

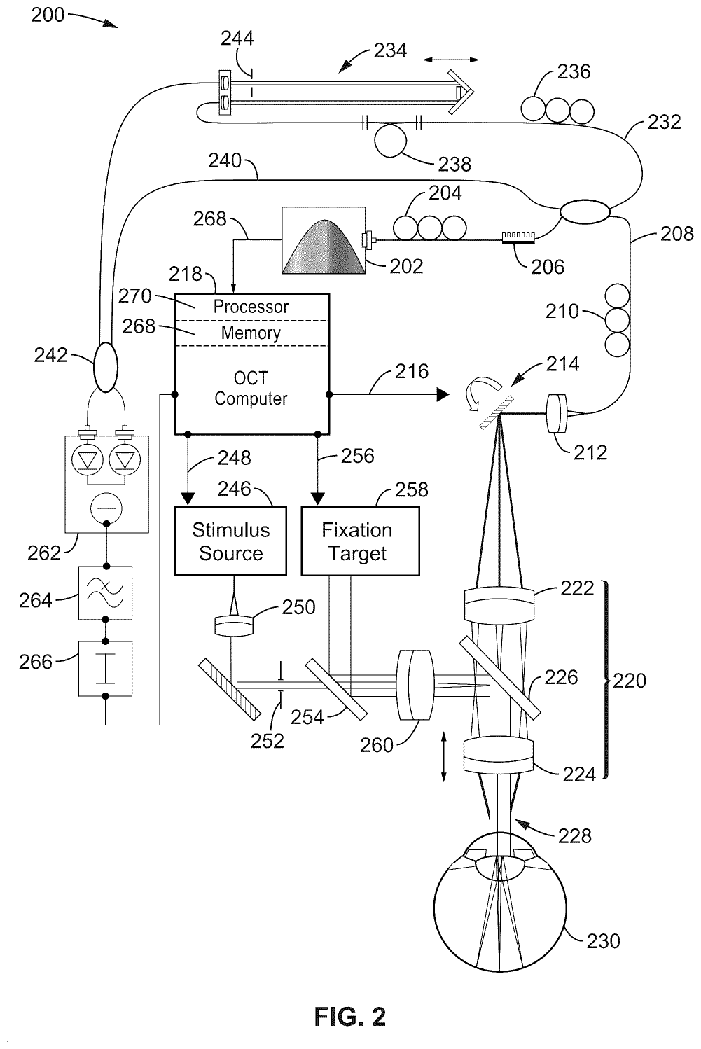

FIG. 2 schematically illustrates an embodiment of our custom optical coherence tomography (OCT) system 200. In the embodiment shown, the system employs a 1060 nm swept-source 202 (e.g., SSOCT; Axsun; Billerica, MA, USA) having a 100 kHz A-scan rate and 100 nm bandwidth. The light output from the swept-source is directed through a polarization controller 204 to a fiber Bragg grating (FBG) 206 that generates a notch in the acquired spectra which is used for spectral alignment of scans. Ten percent 208 of the light from the swept-source 202 is directed through a polarization controller 210 to the sample arm, where it is collimated by a collimator 212 (e.g., AC080-10-B; Thorlabs; Newton, NJ, USA) before passing through the galvanometric scanners 214 (e.g., 6210H & 8310K; Cambridge Technology; Bedford, MA, USA) controlled by scanner signals 216 from the OCT computer 218. The light then passes through a de-magnifying telescope 220 (f_{L1}=100 nm, f_{L2}=75 mm) comprising lens 222, lens 224 and beamsplitter 226, thus creating a 1.2 mm diameter beam 228 on the cornea of the eye 230, near the eye's pupil plane. Lens 224 can be translated along the optical axis to correct defocus. Ninety percent 232 of the swept-source light is directed to the reference arm 234 through a polarization controller 236 and dispersion-compensating fiber patch cord 238. The backscattered light 240 from the eye is combined with the reference arm using a 50/50 fiber coupler 242. The reference arm power can be adjusted using an aperture 244 and a retroreflector can be translated in one dimension to adjust the reference arm length. In our experiments, the optical power for the OCT in the sample arm at the pupil plane was measured to be 1.8 mW.

To deliver a temporally controlled stimulus flash to the retina, a fiber-coupled 555 nm light emitting diode 246 (e.g., MINTF4; Thorlabs; Newton, NJ, USA) controlled by stimulus trigger signals 248 from OCT controller 218 is used. Light exiting the fiber is collimated by a collimator 250 and filtered using a 23 nm bandpass filter centered at 555 nm (e.g., FF01-554/23; Semrock; Lake Forest, IL, USA). To provide a fixation target for the subject, a projector (e.g., Kodak RODPJS75; Eastman Kodak Company; Rochester, NY, USA) projects an image of a star onto the ceiling 258. The position of the target is controlled by signals 256 from the OCT computer 218, such that acquired images and fixation locations are stored together. The stimulus light passes through an aperture 252 and is combined with the fixation target light using a pellicle beamsplitter 254 (e.g., NPX9 50/50; National Photocolor; Mamaroneck, NY, USA). The stimulus light and fixation light are combined with the OCT beam using a dichroic beam splitter 226 (e.g., T715lp; Chroma; Bellows Falls, VT, USA), and the combination passes through a non-magnifying telescope (f_{L3,L2}=75 mm) comprising lens 260 and lens 224 before entering the eye. In our experiments, the diameter of the stimulus was approximately 360 μm on the retina, smaller than the imaged region. The mismatch arose because the light source was powerful enough to bleach only 15% of photopigment in a circular region with a diameter of 750 μm. The 555 nm stimulus wavelength was selected because it isomerizes L- and M-photopigments equally, which make up the overwhelming majority of cones.

The voltage signal from the balanced detector 262 (e.g., PDB481C-AC; Thorlabs; Newton, NJ, USA) is filtered with a 150 MHz low pass filter 264 (e.g., SLP-150+; Mini-Circuits; Brooklyn, NY, USA) and attenuated by a 15 dB attenuator 266 (e.g., VAT-15+; Mini-Circuits; Brooklyn, NY, USA) before being sampled at 12 bits with a ±400 mV range using a digitizer (e.g., ATS9350; AlazarTech; Pointe-Claire, QC, Canada) in the OCT computer. The swept-source provides timing signals 268 from its k-clock and sweep trigger to the digitizer. The OCT computer uses the k-clock signal to sample the OCT fringe at the correct points. The analog waveforms to control the galvanometric scanners 214 are generated using a multifunction data acquisition card (e.g., NI6251; National Instruments; Austin, TX, USA), and the OCT data acquisition is synchronized with the waveform generation.

The general OCT operational software is described in the literature. When the OCT acquisition starts, a 5 V trigger pulse is sent to a function generator (e.g., DG4202; RIGOL; Suzhou, JS, China), which then sends the preconfigured stimulus signal to the LED controller (e.g., DC4100; Thorlabs; Newton, NJ, USA). By setting the delay, width, and the amplitude of this signal, the stimulus flash can be modified to acquire the desired number of pre-stimulus (control) B-scans and to bleach the desired percentage of photopigment.

### 3. Example—Calculating Photopigment Bleaching

Photopigment bleaching can be calculated using the equation

$$p ( t ) = {A}_{0} {e}^{- \frac{T}{{Q}_{e}} t} , \qquad ( 1 )$$

where A₀ is the initial amount of the photopigment, p(t) is the fraction of the remaining photopigment, T is the conventional illuminance of the stimulus flash in Trolands (Td), t is the duration of the stimulus (in seconds) and Qe is the conventional luminous exposure (in Td·sec) needed to deplete the photopigment to 1/e. We used the value of 2.4×10⁶ Td·sec for Qe. The conventional retinal illuminance T can be calculated from the luminous power using the following equation:

$$T = {P}_{v} {Ω}^{- 1} \times 1 {0}^{6} , \qquad ( 2 )$$

where Pᵥ is the photopic luminous power in lumens and Ω is the solid angle measured from the nodal point of the eye. To convert our radiometric measurement into photometric units, we use:

$$P v = K m {\int }_{λ 1}^{λ 2} P e ( λ ) V ( λ ) d λ , \qquad ( 3 )$$

where Kₘ is a conversion constant equal to 683 lm/W, Pₑ is the radiant power and V(λ) is the photopic luminous efficiency function. For simplicity, we assumed our light to be monochromatic with λ_{c}=555 nm, resulting in V(λ) of 1. Finally, by combining equations (2) and (3), we get:

$$T = 683 \times {P}_{e} {Ω}^{- 1} \times 1 {0}^{6} = 683 \times {{P}_{e} ( \frac{A}{{r}^{2}} )}^{- 1} \times 1 {0}^{6} , \qquad ( 4 )$$

where the unit of Pₑ is the radiant power in watts, A is the illuminated area, and r is the distance from the eye's nodal point to the retina (which we assumed to be 17 mm). As the stimulus size on the retina has a diameter of 360 μm, we have all the information needed to convert the optical power measured from the pupil plane into Trolands, and then use it in equation (1). The resulting bleaching levels and corresponding optical powers are listed in Table 1.

### 4. Example—Imaging Protocol

The use of mydriatic drops was not necessary. A custom bite bar was fabricated for each subject to position and stabilize their head. For ORG measurements, subjects were dark adapted for five minutes and then asked to look into the system and fixate on the fixation target 258 (FIG. 2). The OCT system was configured to scan in one dimension only, acquiring a series of B-scans in a single location. B-scans consisting of 250 A-scans (scan width approximately 750 μm on the retina) were collected at a rate of 400 Hz. B-scans were collected before and after the stimulus flash, with the 40 scans before and after the flash used for subsequent analysis. The stimulus size was controlled by an aperture and set to 3.5 mm at the pupil-conjugate plane, intended to prevent clipping by the iris and allow all the stimulus power to reach the retina.

Two experimental parameters were explored: distance from the foveal center and stimulus dose. For distance, a total of twenty retinal loci were imaged after each period of dark adaptation. The multiple loci lay in concentric, iso-eccentric rings, with measurements taken at five loci for each distance. For the dose-dependency, the stimulus power was changed after each dark adaptation round.

All measurements were done on the temporal side of the retina at distances of 2°, 4°, 6° and 8° away from the foveal center. A total of three different subjects were used in this study, all having healthy maculae. Images were acquired at each of the four distances from the fovea, in all three subjects. In one of the subjects, the stimulus dose was varied to achieve six different L/M-photopigment bleaching levels, between 0% and 66%, with ORG measurements collected at four retinal eccentricities for each bleaching level.

### 5. Signal Processing

Our custom signal processing takes place in the OCT computer 218 using signal processing instructions that are stored in a memory 268 for execution on a processor 270. The signal processing can be implemented using, for example, a standalone signal processing unit that is operatively connected to the OCT computer, a signal processing unit that is integrated into the OCT computer, or customized OCT software. Accordingly, it will be appreciated that the OCT computer in combination with this signal processing can serve as special-purpose machine configured for performing the signal processing and other functions described herein.

In one embodiment, the recorded signal is processed in two stages as follows.

In the first stage, the raw spectral data is converted into cross-sectional OCT images (B-scans), using well-established approaches in the OCT literature. In short, raw digitized spectra are aligned using the FBG notch to create phase-stable B-scans. After the alignment, DC-bias is estimated and removed. The spectra are then corrected for dispersion mismatch between the two interferometer arms using numerical dechirping, and complex-valued B-scans are generated by Fourier transforming the processed spectra. The B-scans are also flattened, such that the IS/OS and COST reflections lay at the same height for each A-scan in the image. Flattening is performed by linearly shearing the B-scan until the height of the IS/OS and COST peaks in the laterally averaged B-scan is maximized.

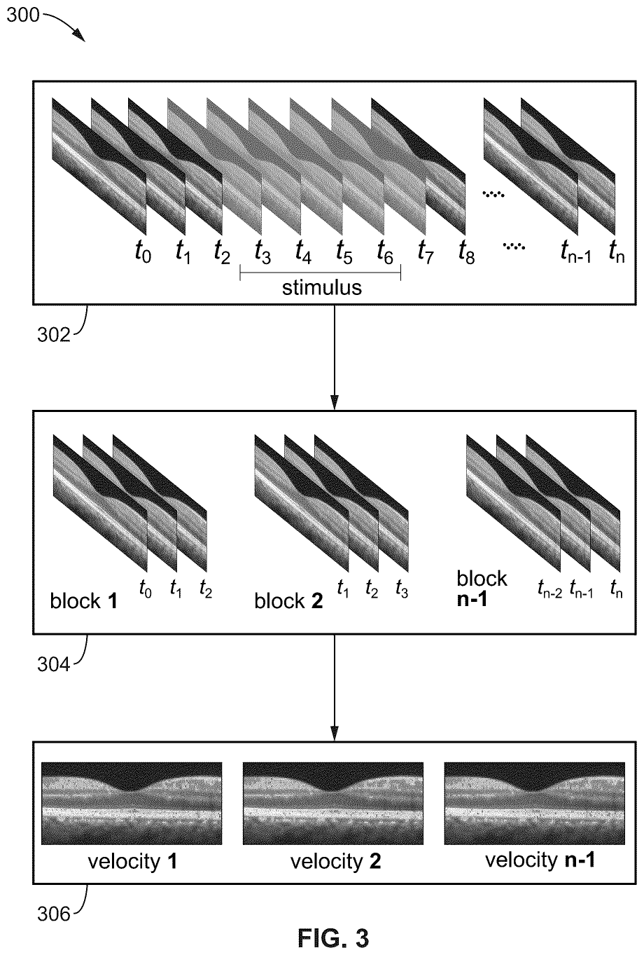

In the second stage, the ORG signal is extracted. One embodiment of the extraction process 300 is illustrated in FIG. 3 which shows the top-level data processing pipeline. In general terms, once the block size width is selected, the phase slopes are obtained from the block data as it moves with a step size of one B-scan through the acquired dataset.

Starting with serially-acquired B-scans 302, in one embodiment the B-scans are divided into temporally overlapping blocks 304 (block 1, block 2, . . . block n−1 are shown). Next, registration, bulk motion estimation, phase unwrapping, and slope calculation 306 are performed to yield corresponding velocities (velocity 1, velocity 2, . . . velocity n−1 are shown).

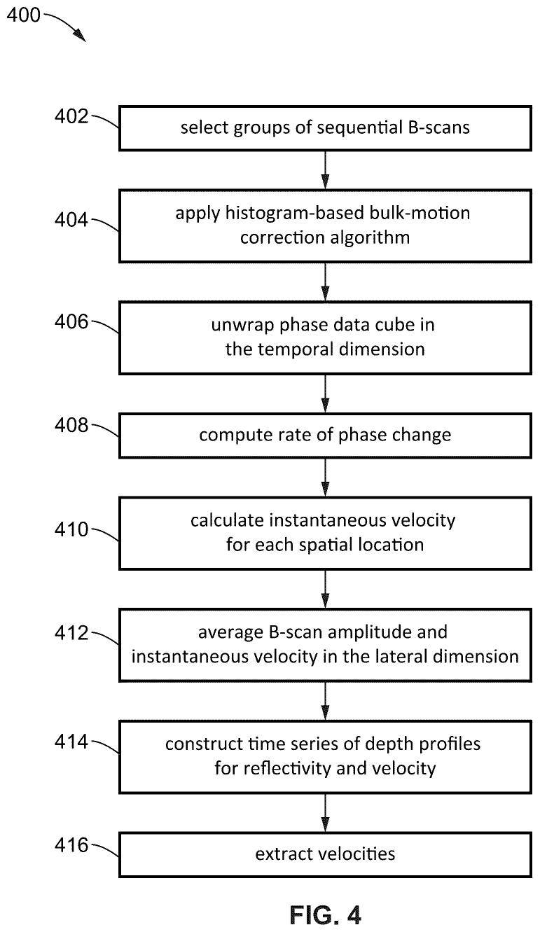

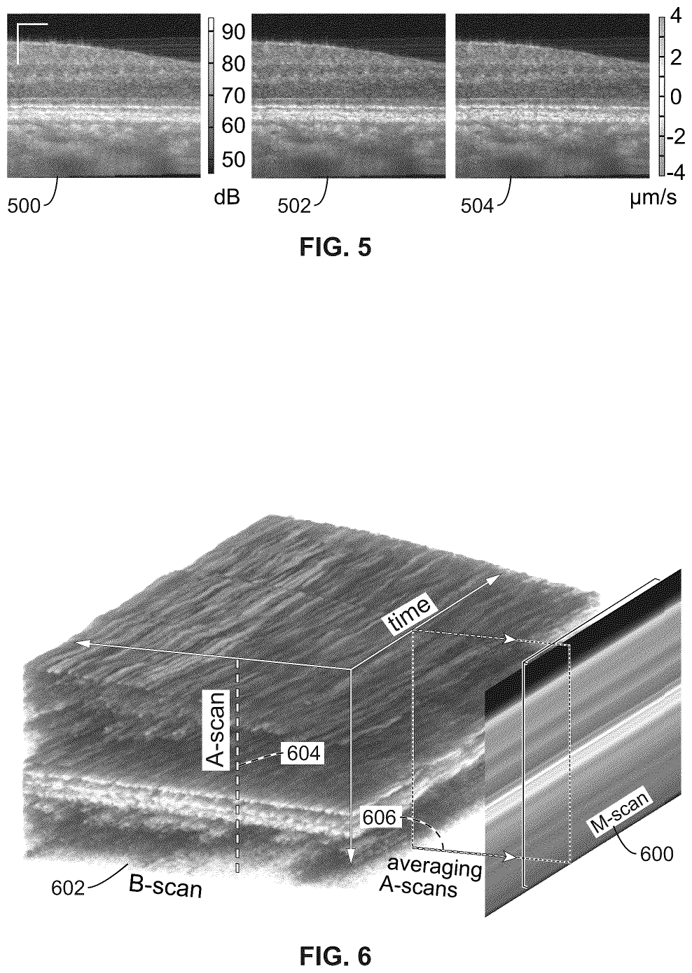

More specifically, referring to the process flow 400 illustrated in FIG. 4, in one embodiment the B-scans are converted into estimates of tissue velocity as follows. First, at step 402, a moving 10 ms time window is used to select groups of five sequential B-scans at a time. Next, at step 404, a histogram-based bulk-motion correction algorithm is utilized to compensate for the axial eye movement during the 10 ms interval, with motion corrected relative to the first B-scan in the series. After bulk-motion correction, the resulting complex data cube (V) may be described as:

$$V ( x , z , t ) = A ( x , z , t ) {e}^{j θ ( x , z , t )} , \qquad ( 5 )$$

where x and z are the lateral and depth coordinates, respectively, and t is time within the window, i.e., 0 ms≤t≤10 ms. At step 406, the phase data cube θ(x,z,t) is unwrapped in the temporal dimension by adding or subtracting 2π to θ(xₚ,z_{q},tᵣ) in order to minimize |θ(xₚ,z_{q},tᵣ)−θ(xₚ,z_{q},tᵣ₋₁)|, where t, and tᵣ₋₁ represent consecutive phase B-scans. This step is performed for each spatial coordinate pair (xₚ,z_{q}) in the volume. After unwrapping, at step 408 a rate of phase change is computed for each coordinate pair by performing a least-squares linear fit with respect to t, giving Δθ/Δt(x,z) in rad/s. From this, the instantaneous velocity for each spatial location is calculated at step 410 according to:

$$\frac{Δ z}{Δ t} ( x , z ) = \frac{Δ θ}{Δ t} ( x , z ) \cdot \frac{λ}{4 π n} , \qquad ( 6 )$$

where λ=1060 nm and n=1.38, the nominal refractive index of the eye. An amplitude B-scan 500, along with overlays of instantaneous pre-stimulus velocities 502 and post-stimulus velocities 504 are shown in FIG. 5.

At step 412, the next step in quantifying the response is to average both the B-scan amplitude and

$$( \frac{Δ z}{Δ t} ( x , z ) )$$

in the lateral dimension, giving instantaneous, depth-dependent measures of backscattering and velocity

$$( \frac{Δ z}{Δ t} ( z ) ) ,$$

respectively. At step 414, by shifting the 10 ms window by one B-scan period (2.5 ms) at a time, a time series of depth profiles can be constructed, separately for reflectivity and velocity. Then, at step 416, the velocities are extracted.

Referring also to FIG. 6, both of these can be visualized in time-depth coordinates, as M-scans 600. FIG. 6 also shows the B-scan 602, A-scan 604, and averaging A-scans 606. For visualization of their velocities, it can be helpful to exclude the velocities of the dimmest parts such as the nuclear layers and ganglion cell layer, where calculated velocities may have been dominated by phase noise instead of movement. From

$$( \frac{Δ z}{Δ t} ( z , t ) ) ,$$

the velocities of the IS/OS and COST layer movements are extracted, and the difference between them is the velocity of contraction/elongation of the OS in the region, which we term v_{OS}(t) or simply v(t) hereafter.

### 6. Experimental Results

For each experimental condition, multiple measurements of v(t) were acquired. The time-varying mean and standard error of the mean (SEM) were calculated from these measurements and used to generate plots. Because v(t) is derived from a block of B-scans, its value is the convolution of the instantaneous velocity with a block-sized rectangular function, which may lead to underestimates of the magnitude of the true velocity. This underestimation may be corrected through numerical deconvolution, since the size and shape of the convolution kernel are known exactly.

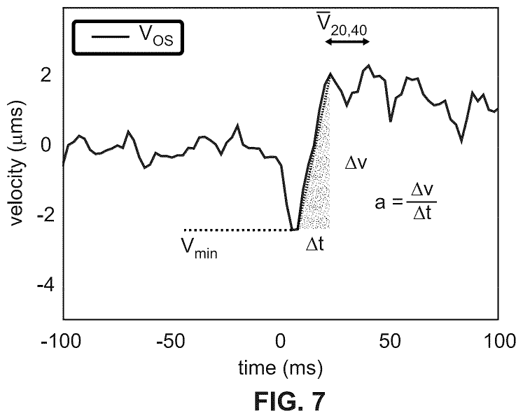

Preliminary examination of the data revealed the following biphasic response: an initial OS contraction (v(t)<0) over the first 10 ms, followed by an elongation (v(t)>0) that appeared to be somewhat stable between 20 and 40 ms. Research use and clinical translation of the present technology requires quantitative/numerical summarization of the ORG responses. Examples of such figures of merit are illustrated in FIG. 7: the most negative velocity after stimulus (vₘᵢₙ), the greatest positive acceleration (aₘₐₓ), and the time-averaged velocity between 20 and 40 ms after stimulus (v̅_{20,40}). A virtue of these figures of merit is that they depend on data collected within 40 ms of the stimulus flash, thereby avoiding corruption by reflexes caused by the stimulus, such as blinks and saccades. For all measurements, the OS length was recorded as well. For assessment by physicians, and for comparison to normative data, numerical summaries of the ORG response may be preferable to the qualitative visualizations described above. Parameters including, but not limited to, (vₘᵢₙ), (aₘₐₓ), and (v̅_{20,40}) may serve as useful clinical figures of merit.

To compare the present measurements of OS velocity to earlier ORG measurements, we reconstructed the OS length response by numerical integration of velocity. ORG responses described above were computed by laterally averaging the phase velocities of the IS/OS and COST layers, which creates a trade-off between SNR and resolution. To quantify this trade-off, the ORG (signal/SNR) was quantified as a function of the extent of lateral averaging in the images.

We observed that in the first five milliseconds after bleaching, the IS/OS velocity becomes positive (corresponding to downward movement, in the M-scan) while the COST velocity becomes negative (corresponding to upward movement). The movement of these features toward one another is consistent with the contraction of the OS. Over the subsequent ten milliseconds, the velocities of the two layers reverse, with the IS/OS moving upward and the COST moving downward, consistent with OS elongation. When the velocity of these two layers is subtracted, the end result is the rate of OS length change (v_{OS}). The results that we observed were consistent with previous reports using adaptive optics, which showed that the OS contracts initially (<10 ms after stimulation) and elongates after that for >100 milliseconds.

Velocity responses were measured at different eccentricities in three different healthy subjects. A response was visible in all individual trials and could be seen in the average response across all subjects and eccentricities. Both the contractile and elongation periods appeared to scale with eccentricity, reducing toward the periphery in all three subjects. In many individual trials, velocity measurements became noisy around 70 ms after the stimulus flash. This led us to believe that this noise is a consequence of a reflexive eye movement that reduces the effectiveness of our current bulk-motion algorithm. Since the following statistical analysis utilized B-scans collected within 40 ms of the stimulus onset, they were unaffected by this noise.

Additionally, the eccentricity-dependence of the response was observed. The most negative velocity, which corresponded to the largest rate of contraction, decreased in magnitude between 2° and 6°, with similar values at 6° and 8°. The maximum positive acceleration decreased in magnitude between 2° (1.5 to 2.1 μm/s²) and 6° (1.1 to 1.5 μm/s²).

Dose-dependence of the response was also observed. Contractile velocity was very similar at doses of 0% and 4% but appeared to be different in the case of elongation velocity. When the elongation phase was quantified as the average velocity between 20 and 40 milliseconds post-stimulus (v_{20,40}), a similar trend was visible, with brighter stimuli resulting in faster elongation. In response to the brightest stimuli (66% bleaching), elongation velocities were in the range of 1.00 to 1.25 μm/s. Qualitatively, dose and response appeared to have a log-linear relationship between 4% and 66% bleaching.

We also performed numerical integration of the OS velocity responses. The velocity of the rapid elongation stage, between approximately 10 and 100 ms, appeared to be about 1 μm/s. This was substantially lower than previous studies, which reported a velocity of approximately 3 μm/s to a 70% bleach and velocities approaching 3.9 μm s and 4.6 μm/s for similar flashes. We note that numerical integration is not necessary for this comparison, as the slope of the integrated curve is the same as the time-averaged velocity in the same 10 to 100 ms interval.

### 7. Discussion

It will be appreciated that the non-AO optoretinographic method described above depends on the correlated movement of neighboring tissue, and thus circumvents the need for cellular resolution and tracking. Compared to more advanced techniques, this method offers a simpler imaging system, reduced data sizes, faster signal processing, and smaller demands on personnel. One of its key advantages is potentially straightforward incorporation into existing commercial OCT technology. We were able to acquire data in approximately ten minutes per subject, including dark adaptation, and only one operator was required. Results can be generated within minutes, making the approach attractive to clinicians and large-scale studies.

The main result of this method, namely, a time series of OS velocity, can be integrated to approximate the results of earlier, position-based optoretinographic methods. Congruity between the two results is an advantage to both, as they can contribute to the same growing body of literature and data. Our experimental results revealed velocities that are lower by a factor of two to three than earlier position-based results. The pattern of contraction and elongation demonstrated here bears significant similarity to earlier results, including the timing of the early and late stages. This suggests that the biophysical processes underlying the responses are the same. The reason for the discrepancy in velocities is not known but could potentially be due to optical factors such as the role played by the lateral PSF size and contributions of portions of the tissue that might be stationary. We contemplate that such systematic differences between the approaches can be resolved by calibration.

The clear relationship between ORG responses and distance from the fovea may be due to differences in outer segment anatomy, and normalizing by OS length seems to weaken this relationship. When designing the imaging protocol, we strove to avoid realignment of the optical system with changes in fixation. Given that, and the fact that this method does not require dilation of the pupil, there is a risk that the subject's iris could clip the (e.g., 4 mm diameter) stimulus beam after changing fixation. We did not actively monitor this, but it could be done with a real-time pupil camera programmed to detect iridial reflexes. Alternatively, a brighter stimulus source could be used, permitting the beam diameter at the pupil to be smaller.

If the OCT and stimulus beam diameters were the same, the OCT signal itself could be used to calculate the stimulus efficiency (not withstanding chromatic differences). Moreover, because the trends were similar among subjects, regardless of which fixation target location was used for initial alignment, we do not believe that the stimulus beam was clipped.

A distinct trend was also observed when we altered the stimulus energy. Photopigment bleaching is dependent on stimulus dosage between 8% and 66% pigment bleaching values. Establishing dose-dependence is a critical step in developing novel functional assays. The dose-dependence observed here was consistent with similar observations of dose-dependence using position-based optoretinographic methods, providing further assurance of the interoperability of these two methods.

We observed that optoretinograms were reliably produced from all tested eccentricities when the stimulus energy bleached more than 8% of the photopigment. While this sensitivity is lower than what has been reported with AO-OCT systems, the noise floor of the present method could potentially be reduced with improved bulk-motion correction. The 30 ms duration of the stimulus flash was required to reach a photopigment bleaching rate of 66%, due to limitations in the source power and our optical design. A more powerful source and redesign of the stimulus channel could permit shorter, more intense flashes, which would likely improve the method's sensitivity. Results which were observed to be non-zero even in the absence of a stimulus flash (0% bleaching) may be a consequence of noise combined with the bias involved in identifying the minimum (most negative) velocity. This bias probably limits the sensitivity of this figure of merit.

One of the motivating assumptions of this method is that there are finite time intervals over which the retina is effectively motionless, i.e., that the axial component of motion is small with respect to the wavelength of the light source and that the lateral component is small with respect to the diffraction-limited spot size (or speckle size). While this assumption appeared largely to be true, evidence of motion artifacts was present in many of the measurements. A potentially powerful way to improve the sensitivity of this approach would be to detect and filter movement artifacts. When analyzing a series of B-scans to measure instantaneous velocity, we monitored the residual least-square error and the correlation of B-scan amplitude. Preliminary investigation showed that both estimates of error were correlated with deviations from expected velocity measurements.

Our results reveal a biphasic ORG response, consisting of an initial contraction and later elongation of the OS. These phases of the response are thought to have different physiological origins and may thus confer distinct clinical benefits. Some researchers, by demonstrating a lack of elongation in mutant mice with a dysfunctional G-protein transducin, suggested that transducing subunit dissociation might drive ORG elongation osmotically. Other researchers hypothesized that the contractile response seen in the ORG is due to the early receptor potential (ERP).

For optoretinograms presented here we used a simple OCT B-scan flattening procedure. However, a segmentation-based approach may be preferable. Adaptive optics studies of photoreceptor morphology have revealed axially staggered locations of IS/OS and COST in neighboring cones. Moreover, the B-scans shown above illustrate that the ideal depth at which to measure the phase of these surfaces can change from A-scan to A-scan. Instead of computing the phase at the same depth for all A-scans, segmentation would permit measurement of phase at the most salient (and brightest) depths, and thereby improve the ORG sensitivity. It would also allow the investigation of eccentricities closer to the fovea, where the thickness of retinal layers can vary substantially within small visual angles. It could potentially help in the imaging of disease-affected retinae as well, where the layers may be sporadically deformed by edema, drusen, or degeneration.

We have shown that it is possible to obtain ORG responses using an SS-OCT system without adaptive optics. The simplicity of the imaging system permits straightforward integration of this method to existing OCT systems.

### 8. Notable Aspects of the Technology

Processing of the ORG signal provides intermediate information from the series of OCT images, distinct from the eventual functional response measurement. This information may be used to improve the accuracy of the ORG recording and reduce its noise, thereby improving its signal-to-noise ratio (SNR) and sensitivity to response differences, e.g., deviations from norms caused by retinal disease. These include, but are not limited to, the following:

### (a) OCT Amplitude

The amplitude of the OCT signal provides a way to measure the impact of eye movements and other sources of noise on groups of OCT B-scans. Correlation of the OCT amplitude over time improves confidence in the resulting ORG signal.

### (b) OCT Phase and Phase Velocity

Phase correlation over time provides an additional estimate of noise and permits further filtering of the ORG signal. Because we now have estimates of normal phase velocity responses at different times after stimulus, significant deviations from the norms may be used to identify motion or other phase artifacts.

### (c) ORG Phase Velocity Fitting Error

When the angular velocity of moving structures is fit with a linear function, the resulting fitting error may be used to filter the ORG signal and improve its SNR and sensitivity.

### (d) Complex Decorrelation

The decorrelation of the complex OCT signal (estimated by variance or standard deviation of the complex signal, and pooled/aggregated spatially over the scanning and depth dimensions) is low when the motion of the retina was low and can be used to determine confidence in each value in the ORG signal.

Results have been shown in this disclosure using a window size of 5 B-scans. In the presence of varying eye movements, the optimal block size may depend on the instantaneous velocity of the retina in lateral and axial dimensions. When the eye is relatively stationary, larger blocks may be useful, whereas shortly before and after eye movements, smaller blocks may be useful. A simple way to assess the optimal series of block sizes is to compute phase velocities (as described above) over every possible set of blocks (i.e., every subset of continuous B-scans within the full set), and also the filtering figures of merit described above. The figures of merit can be used to decide the optimal set of block sizes, thereby further improving the SNR and sensitivity of the ORG signal.

A variety of stimulus patterns have been tested, each with its own virtues. Adapting backgrounds and flicker stimuli can be used to reduce dark-adaptation time, and thus increase the clinical utility of the ORG test. Flicker stimuli can further improve the ORG SNR by permitting analysis of the signal in the frequency domain, where the responses to multiple flashes are aggregated and noise is reduced. Multiple stimuli can be used to test the impacts of dark and light adaptation on the ORG response, thereby providing access to additional biomarkers (viz., dark and light adaptation dynamics). Retinal neurons other than the photoreceptors may be interrogated using stimuli designed to maximize their response (e.g., contrast-reversing checkerboard and “dead leaves” stimuli known to isolate and maximize the response from ganglion cells).

While the 400 Hz B-scan rate shown in the included results is sufficient for the measurement of ORG responses, other rates may provide distinct advantages. Slower scanning rates can improve the OCT SNR and thus the sensitivity to phase changes, permitting measurement of responses to dimmer stimuli. Faster scanning rates can reduce the impact of eye movements within the ORG blocks.

For testing this method, we were limited to a 100 kHz swept-source laser, similar in speed to existing commercial OCT lasers. However, laser technology continues to improve, and faster OCT systems are on the horizon. Implementation of this method using faster lasers may result in improved ORG performance, with the specific possible benefits of improved phase noise rejection and ORG SNR.

Instead of serial B-scan imaging, small volumes may be acquired instead, where a slow-axis scanner is scanned over an extent of retina smaller than that of the fast-axis scanner. Such volumes could be used to correct eye movements in the slow-axis dimension, and thus to reduce or remove the impact of “out of plane” movements, where the retinal tissue being imaged moves out of the plane of the imaging scanner.

In addition to the main approach outlined in this disclosure, various statistical approaches may be used instead. The main approach is mechanistic, in that the phenomenon to be quantified is a specific mechanism; namely the rate of light-evoked changes in cell size. However, in cases where the retinal structure is not well-organized, blunter statistical methods may be called for. The statistical dispersion of phase, aggregated spatially, is an example of such a method. For instance, after the numerical correction of bulk-motion phase artifacts, the variance of the complex OCT signal (or its amplitude or its phase) with respect to time could be used to quantify the instantaneous activity of the retinal tissue. This variance increases in response to light stimuli and exhibits dose dependence. While it may have a limited dynamic range compared to mechanistic figures of merit, it has the advantage of obviating location, segmentation, and correct labeling of retinal features. This advantage may be key in assessing functional responses in retinae affected by the disease.

There have been no published reports of using scanning OCT to measure responses in post-receptoral retinal neurons, but in principle, all of the methods outlined here could be used to measure responses in bipolar cells and retinal ganglion cells, either in the layers containing their nuclei (the inner nuclear layer and ganglion cell layer), their synapses (the outer and inner plexiform layers), or their fibers (the nerve fiber layer).

Furthermore, implementations of our technology may include, without limitation, one or more of the following:
1. Oversampling the lateral point spread function of the optical system by a factor of about 3. The lateral PSF in our system is ˜18 microns, which would suggest a measurement interval of 9 microns to achieve Nyquist sampling. We collect A-scans every 3 microns, effectively oversampling by a factor of 3.
2. Acquiring serial B-scans. Because our laser is not fast enough to acquire volumetric images at rates sufficient to measure the ORG, we acquire serial B-scans instead. This limitation posed the key challenge to development of the velocity-based approach. We assume that the eye moves during the course of our measurement, and we assume that the correlated behavior of cells in the stimulated region will indicate the response in that area.
3. Not needing to track the retina's movement in the lateral dimensions over time.
4. Using a B-scan rate of 400 Hz which is substantially slower than the B-scan rates of (or effective B-scan rates) state-of-the-art, experimental OCT approaches. In spite of this, by limiting ourselves to serial B-scan imaging, we achieve ORG bandwidth (400 Hz) higher than any of the conventional AO or DAC approaches.
5. Using a field of view of 2.5 degrees, which is substantially smaller than that of commercial systems, in order to improve our sampling density and our B-scan rate.
6. Employing a fiber Bragg grating to produce phase-stable A-scans (i.e., spectral lines that can be aligned with sub-nanometer precision).
7. Scanning at 400 Hz, which is substantially faster than ordinary commercial systems.
8. Sampling the retina more densely than commercial OCT systems. Our field of view is 750 microns (2.5 degrees) and we acquire 250 scans over that region.
9. Incorporating a visible stimulus channel, capable of delivering flash stimuli (and other stimuli) to the portion of the retina being imaged.

From the description herein, it will be appreciated that the present disclosure encompasses multiple implementations of the technology which include, but are not limited to, one or more of the following:

A method for velocity based optoretinography (ORG), the method comprising: (a) providing an optical coherence tomography (OCT) system; (b) applying an optical stimulus to an eye of a subject; (c) assessing tissue velocity of outer segment tips and inner segment/outer segment junctions of the eye in response to said stimulus with optoretinography (ORG); and (d) correlating stimulus-evoked tissue velocities with a healthy or diseased state.

A method for velocity based optoretinography (ORG), the method comprising: (a) obtaining raw spectral data from scanning an eye of a subject; (b) transforming the raw spectral data into serially-acquired optical coherence tomography (OCT) B-scans; (c) dividing the B-scans into temporally overlapping blocks; and (d) performing registration, bulk motion estimation, phase unwrapping, and slope calculation on the blocks to yield corresponding velocities.

The method of any preceding implementation, further comprising: (a) selecting groups of sequential B-scans using a moving time window; (b) using a histogram-based bulk-motion correction algorithm to compensate for axial eye movement during the time window, wherein motion is corrected relative to the first B-scan in the series, and wherein after bulk-motion correction, a resulting complex data cube (V) may be described as V(x,z,t)=A(x,z,t)e^{jθ(x,z,t)}, where x and z are the lateral and depth coordinates, respectively, and t is time within the window; (c) unwrapping the phase data cube θ(x,z,t) in the temporal dimension by adding or subtracting 2π to θ(zₚ,z_{q},tᵣ) in order to minimize |θ(xₚ,z_{q},tᵣ)−θ(xₚ,z_{q},tᵣ₋₁)|, where t, and tᵣ₋₁ represent consecutive phase B-scans, wherein this step is performed for each spatial coordinate pair (xₚ,z_{q}) in the volume; (d) computing a rate of phase change is computed for each coordinate pair by performing a least-squares linear fit with respect to t, giving Δθ/Δt(x,z) in rad/s; (e) calculating the instantaneous velocity for each spatial location is calculated according to:

$$\frac{Δ z}{Δ t} ( x , z ) = \frac{Δ θ}{Δ t} ( x , z ) \cdot \frac{λ}{4 π n} ,$$

where λ=1060 nm and n=1.38, the nominal refractive index of the eye; (f) averaging both the B-scan amplitude and

$$( \frac{Δ z}{Δ t} ( x , z ) )$$

in the lateral dimension, giving instantaneous, depth-dependent measures of backscattering and velocity

$$( \frac{Δ z}{Δ t} ( z ) ) ,$$

respectively; (g) shifting the time window by one B-scan period at a time, and constructing a time series of depth profiles, separately for reflectivity and velocity; (h) visualizing the depth profiles in time-depth coordinates, as M-scans; and (i) assessing tissue velocities from the M-scans.

A method for velocity based optoretinography (ORG), the method comprising: (a) obtaining optical coherence tomography (OCT) B-scans of an eye of a subject; (b) selecting groups of sequential B-scans using a moving time window; (c) using a histogram-based bulk-motion correction algorithm to compensate for axial eye movement during the time window, wherein motion is corrected relative to the first B-scan in the series, and wherein after bulk-motion correction, a resulting complex data cube (V) may be described as: V(x,z,t)=A(x,z,t)e^{jθ(x,z,t)}, where x and z are the lateral and depth coordinates, respectively, and t is time within the window; (d) unwrapping the phase data cube θ(x,z,t) in the temporal dimension by adding or subtracting 2π to θ(xₚ,z_{q},tᵣ) in order to minimize |θ(xₚ,z_{q},tᵣ)−θ(xₚ,z_{q},tᵣ₋₁)|, where t, and tᵣ₋₁ represent consecutive phase B-scans, wherein this step is performed for each spatial coordinate pair (xₚ,z_{q}) in the volume; (e) computing a rate of phase change is computed for each coordinate pair by performing a least-squares linear fit with respect to t, giving Δθ/Δt(x,z) in rad/s; (f) calculating the instantaneous velocity for each spatial location is calculated according to:

$$\frac{Δ z}{Δ t} ( x , z ) = \frac{Δ θ}{Δ t} ( x , z ) \cdot \frac{λ}{4 π n} ,$$

where λ=1060 nm and n=1.38, the nominal refractive index of the eye; (g) averaging both the B-scan amplitude and

$$( \frac{Δ z}{Δ t} ( x , z ) )$$

in the lateral dimension, giving instantaneous, depth-dependent measures of backscattering and velocity

$$( \frac{Δ z}{Δ t} ( z ) ) ,$$

respectively; (h) shifting the time window by one B-scan period at a time, and constructing a time series of depth profiles, separately for reflectivity and velocity; (i) visualizing the depth profiles in time-depth coordinates, as M-scans; and (j) assessing tissue velocities from the M-scans.

The method of any preceding implementation, wherein the assessment of tissue velocity further comprises measuring the size of retinal substructures.

The method of any preceding implementation, wherein the tissues assessed comprise at least one of cone outer segment tips (COST), rod outer segment tips (ROST), and inner segment/outer segment junctions (IS/OS).

The method of any preceding implementation, wherein said assessing tissue velocity comprises: (a) obtaining a 2-dimensional profile of retinal tissues from B-scans of the subject at multiple time points; (b) assembling the B-scans into a complex M-scan having amplitude and phase that manifest motion of a tissue section; and (c) assessing tissue velocities from the M-scan.

The method of any preceding implementation, wherein said assessing tissue velocity comprises: (a) serially acquiring a plurality of B-scans from the Oct system; (b) dividing the B-scans into temporally overlapping blocks; and (c) performing registration, bulk motion estimation, phase unwrapping, and slope calculation to yield corresponding tissue velocities.

The method of any preceding implementation, further comprising outputting one or more visualizations of the tissue velocities for correlating stimulus-evoked tissue velocities with a healthy or diseased state.

The method of any preceding implementation, further comprising outputting one or more numerical summaries of the tissue velocities for correlating stimulus-evoked tissue velocities with a healthy or diseased state.

An apparatus for velocity based optoretinography (ORG), the apparatus comprising: (a) an optical coherence tomography (OCT) system configured to apply an optical stimulus to an eye of a subject; and (b) a signal processing unit comprising a processor and a non-transitory memory storing instructions executable by the processor to perform steps comprising: (b)(i) assessing tissue velocity of outer segment tips and inner segment/outer segment junctions of the eye in response to said stimulus with optoretinography (ORG); and (b)(ii) correlating stimulus-evoked tissue velocities with a healthy or diseased state.

An apparatus for velocity based optoretinography (ORG), the apparatus comprising: (a) an optical coherence tomography (OCT) system configured to apply an optical stimulus to an eye of a subject, obtain raw spectral data from scanning the eye, and transforming the raw spectral data into serially-acquired optical coherence tomography (OCT) B-scans; and (b) a signal processing unit comprising a processor and a non-transitory memory storing instructions executable by the processor to perform steps comprising: (b)(i) dividing the B-scans into temporally overlapping blocks; and (b)(ii) performing registration, bulk motion estimation, phase unwrapping, and slope calculation on the blocks to yield corresponding velocities.

The apparatus of any preceding implementation, wherein said signal processing unit further performs steps comprising: (a) selecting groups of sequential B-scans using a moving time window; (b) using a histogram-based bulk-motion correction algorithm to compensate for axial eye movement during the time window, wherein motion is corrected relative to the first B-scan in the series, and wherein after bulk-motion correction, a resulting complex data cube (V) may be described as: V(x,z,t)=A(x,z,t)e^{jθ(x,z,t)}, where x and z are the lateral and depth coordinates, respectively, and t is time within the window; (c) unwrapping the phase data cube θ(x,z,t) in the temporal dimension by adding or subtracting 2π to θ(xₚ,z_{q},tᵣ) in order to minimize |θ(xₚ,z_{q},tᵣ)−θ(xₚ,z_{q},tᵣ₋₁), where tᵣ and tᵣ₋₁ represent consecutive phase B-scans, wherein this step is performed for each spatial coordinate pair (xₚ,z_{q}) in the volume; (d) computing a rate of phase change is computed for each coordinate pair by performing a least-squares linear fit with respect to t, giving Δθ/Δt(x,z) in rad/s; (e) calculating the instantaneous velocity for each spatial location is calculated according to:

$$\frac{Δ z}{Δ t} ( x , z ) = \frac{Δ θ}{Δ t} ( x , z ) \cdot \frac{λ}{4 π n} ,$$

where λ=1060 nm and n=1.38, the nominal refractive index of the eye; (f) averaging both the B-scan amplitude and

$$( \frac{Δ z}{Δ t} ( x , z ) )$$

in the lateral dimension, giving instantaneous, depth-dependent measures of backscattering and velocity

$$( \frac{Δ z}{Δ t} ( z ) ) ,$$

respectively; (g) shifting the time window by one B-scan period at a time, and constructing a time series of depth profiles, separately for reflectivity and velocity; (h) visualizing the depth profiles in time-depth coordinates, as M-scans; and (i) assessing tissue velocities from the M-scans.

An apparatus for velocity based optoretinography (ORG), the apparatus comprising: (a) an optical coherence tomography (OCT) system configured to obtain B-scans of the eye; and (b) a signal processing unit comprising a processor and a non-transitory memory storing instructions executable by the processor to perform steps comprising: (b)(i) selecting groups of sequential B-scans using a moving time window; (b)(ii) using a histogram-based bulk-motion correction algorithm to compensate for axial eye movement during the time window, wherein motion is corrected relative to the first B-scan in the series, and wherein after bulk-motion correction, a resulting complex data cube (V) may be described as: V(x,z,t)=A(x,z,t)e^{jθ(x,z,t)}, where x and z are the lateral and depth coordinates, respectively, and t is time within the window; (b)(iii) unwrapping the phase data cube θ(x,z,t) in the temporal dimension by adding or subtracting 2π to θ(xₚ,z_{q},tᵣ) in order to minimize |θ(xₚ,z_{q},tᵣ)−θ(xₚ,z_{q},tᵣ₋₁)|, where t, and tᵣ₋₁ represent consecutive phase B-scans, wherein this step is performed for each spatial coordinate pair (xₚ,z_{q}) in the volume; (b)(iv) computing a rate of phase change is computed for each coordinate pair by performing a least-squares linear fit with respect to t, giving Δθ/Δt(x,z) in rad/s; (b)(v) calculating the instantaneous velocity for each spatial location is calculated according to:

$$\frac{Δ z}{Δ t} ( x , z ) = \frac{Δ θ}{Δ t} ( x , z ) \cdot \frac{λ}{4 π n} ,$$

where λ=1060 nm and n=1.38, the nominal refractive index of the eye; (b)(vi) averaging both the B-scan amplitude and

$$( \frac{Δ z}{Δ t} ( x , z ) )$$

in the lateral dimension, giving instantaneous, depth-dependent measures of the backscattering and velocity

$$( \frac{Δ z}{Δ t} ( z ) ) ,$$

respectively; (b)(vii) shifting the time window by one B-scan period at a time, and constructing a time series of depth profiles, separately for reflectivity and velocity; (b)(viii) visualizing the depth profiles in time-depth coordinates, as M-scans; and (b)(ix) assessing tissue velocities from the M-scans.

The apparatus of any preceding implementation, wherein size of retinal substructures is measured.

The apparatus of any preceding implementation, wherein the tissues assessed comprise at least one of cone outer segment tips (COST), rod outer segment tips (ROST), and inner segment/outer segment junctions (IS/OS).

The apparatus of any preceding implementation, wherein said assessing tissue velocity comprises: (a) obtaining a 2-dimensional profile of retinal tissues from B-scans of the subject at multiple time points; (b) assembling the B-scans into a complex M-scan having amplitude and phase that manifest motion of a tissue section; and (c) assessing tissue velocities from the M-scan.

The apparatus of any preceding implementation, wherein said assessing tissue velocity further comprises: (a) serially acquiring a plurality of B-scans from the OCT system; (b) dividing the B-scans into temporally overlapping blocks; and (c) performing registration, bulk motion estimation, phase unwrapping, and slope calculation to yield corresponding tissue velocities.

The apparatus of any preceding implementation, wherein the apparatus provides one or more visualizations of the tissue velocities for correlating stimulus-evoked tissue velocities with a healthy or diseased state.

The apparatus of any preceding implementation, wherein the apparatus provides one or more numerical summaries of the tissue velocities for correlating stimulus-evoked tissue velocities with a healthy or diseased state.

Embodiments of the present technology may be described herein with reference to flowchart illustrations of methods and systems according to embodiments of the technology, and/or procedures, algorithms, steps, operations, formulae, or other computational depictions, which may also be implemented as computer program products. In this regard, each block or step of a flowchart, and combinations of blocks (and/or steps) in a flowchart, as well as any procedure, algorithm, step, operation, formula, or computational depiction, can be implemented by various means, such as hardware, firmware, and/or software including one or more computer program instructions embodied in computer-readable program code. As will be appreciated, any such computer program instructions may be executed by one or more computer processors, including without limitation a general purpose computer or special purpose computer, or other programmable processing apparatus to produce a machine, such that the computer program instructions which execute on the computer processor(s) or other programmable processing apparatus create means for implementing the function(s) specified.

Accordingly, blocks of the flowcharts, and procedures, algorithms, steps, operations, formulae, or computational depictions described herein support combinations of means for performing the specified function(s), combinations of steps for performing the specified function(s), and computer program instructions, such as embodied in computer-readable program code logic means, for performing the specified function(s). It will also be understood that each block of the flowchart illustrations, as well as any procedures, algorithms, steps, operations, formulae, or computational depictions and combinations thereof described herein, can be implemented by special purpose hardware-based computer systems which perform the specified function(s) or step(s), or combinations of special purpose hardware and computer-readable program code.

Furthermore, these computer program instructions, such as embodied in computer-readable program code, may also be stored in one or more computer-readable memory or memory devices that can direct a computer processor or other programmable processing apparatus to function in a particular manner, such that the instructions stored in the computer-readable memory or memory devices produce an article of manufacture including instruction means which implement the function specified in the block(s) of the flowchart(s). The computer program instructions may also be executed by a computer processor or other programmable processing apparatus to cause a series of operational steps to be performed on the computer processor or other programmable processing apparatus to produce a computer-implemented process such that the instructions which execute on the computer processor or other programmable processing apparatus provide steps for implementing the functions specified in the block(s) of the flowchart(s), procedure(s) algorithm(s), step(s), operation(s), formula (e), or computational depiction(s).

It will further be appreciated that the terms “programming” or “program executable” as used herein refer to one or more instructions that can be executed by one or more computer processors to perform one or more functions as described herein. The instructions can be embodied in software, in firmware, or in a combination of software and firmware. The instructions can be stored local to the device in non-transitory media, or can be stored remotely such as on a server, or all or a portion of the instructions can be stored locally and remotely. Instructions stored remotely can be downloaded (pushed) to the device by user initiation, or automatically based on one or more factors.

It will further be appreciated that, as used herein, the terms processor, hardware processor, computer processor, central processing unit (CPU), and computer are used synonymously to denote a device capable of executing the instructions and communicating with input/output interfaces and/or peripheral devices, and that the terms processor, hardware processor, computer processor, CPU, and computer are intended to encompass single or multiple devices, single core and multicore devices, and variations thereof.

As used herein, the term “implementation” is intended to include, without limitation, embodiments, examples, or other forms of practicing the technology described herein.

As used herein, the singular terms “a,” “an,” and “the” may include plural referents unless the context clearly dictates otherwise. Reference to an object in the singular is not intended to mean “one and only one” unless explicitly so stated, but rather “one or more.”

Phrasing constructs, such as “A, B and/or C”, within the present disclosure describe where either A, B, or C can be present, or any combination of items A, B and C. Phrasing constructs indicating, such as “at least one of” followed by listing a group of elements, indicates that at least one of these group elements is present, which includes any possible combination of the listed elements as applicable.

References in this disclosure referring to “an embodiment”, “at least one embodiment” or similar embodiment wording indicate that a particular feature, structure, or characteristic described in connection with a described embodiment is included in at least one embodiment of the present disclosure. Thus, these various embodiment phrases are not necessarily all referring to the same embodiment, or to a specific embodiment which differs from all the other embodiments being described. The embodiment phrasing should be construed to mean that the particular features, structures, or characteristics of a given embodiment may be combined in any suitable manner in one or more embodiments of the disclosed apparatus, system or method.

As used herein, the term “set” refers to a collection of one or more objects. Thus, for example, a set of objects can include a single object or multiple objects.

Relational terms such as first and second, top and bottom, upper and lower, left and right, and the like may be used solely to distinguish one entity or action from another entity or action without necessarily requiring or implying any actual such relationship or order between such entities or actions.

The terms “comprises,” “comprising,” “has”, “having,” “includes”, “including,” “contains”, “containing” or any other variation thereof, are intended to cover a non-exclusive inclusion, such that a process, method, article, or apparatus that comprises, has, includes, contains a list of elements does not include only those elements but may include other elements not expressly listed or inherent to such process, method, article, or apparatus. An element proceeded by “comprises . . . a”, “has . . . a”, “includes . . . a”, “contains . . . a” does not, without more constraints, preclude the existence of additional identical elements in the process, method, article, or apparatus that comprises, has, includes, contains the element.

As used herein, the terms “approximately”, “approximate”, “substantially”, “essentially”, and “about”, or any other version thereof, are used to describe and account for small variations. When used in conjunction with an event or circumstance, the terms can refer to instances in which the event or circumstance occurs precisely as well as instances in which the event or circumstance occurs to a close approximation. When used in conjunction with a numerical value, the terms can refer to a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. For example, “substantially” aligned can refer to a range of angular variation of less than or equal to ±10°, such as less than or equal to ±5°, less than or equal to ±4°, less than or equal to ±3°, less than or equal to ±2°, less than or equal to ±1°, less than or equal to ±0.5°, less than or equal to ±0.1°, or less than or equal to ±0.05°.

Additionally, amounts, ratios, and other numerical values may sometimes be presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

The term “coupled” as used herein is defined as connected, although not necessarily directly and not necessarily mechanically. A device or structure that is “configured” in a certain way is configured in at least that way, but may also be configured in ways that are not listed.

Benefits, advantages, solutions to problems, and any element(s) that may cause any benefit, advantage, or solution to occur or become more pronounced are not to be construed as critical, required, or essential features or elements of the technology described herein or any or all the claims.

In addition, in the foregoing disclosure various features may be grouped together in various embodiments for the purpose of streamlining the disclosure. This method of disclosure is not to be interpreted as reflecting an intention that the claimed embodiments require more features than are expressly recited in each claim. Inventive subject matter can lie in less than all features of a single disclosed embodiment.

The abstract of the disclosure is provided to allow the reader to quickly ascertain the nature of the technical disclosure. It is submitted with the understanding that it will not be used to interpret or limit the scope or meaning of the claims.

It will be appreciated that the practice of some jurisdictions may require the deletion of one or more portions of the disclosure after that application is filed. Accordingly, the reader should consult the application as filed for the original content of the disclosure. Any deletion of content of the disclosure should not be construed as a disclaimer, forfeiture or dedication to the public of any subject matter of the application as originally filed.

The following claims are hereby incorporated into the disclosure, with each claim standing on its own as a separately claimed subject matter.

Although the description herein contains many details, these should not be construed as limiting the scope of the disclosure but as merely providing illustrations of some of the presently preferred embodiments. Therefore, it will be appreciated that the scope of the disclosure fully encompasses other embodiments that may become obvious to those skilled in the art.

All structural and functional equivalents to the elements of the disclosed embodiments that are known to those of ordinary skill in the art are expressly incorporated herein by reference and are intended to be encompassed by the present claims. Furthermore, no element, component, or method step in the present disclosure is intended to be dedicated to the public regardless of whether the element, component, or method step is explicitly recited in the claims. No claim element herein is to be construed as a “means plus function” element unless the element is expressly recited using the phrase “means for”. No claim element herein is to be construed as a “step plus function” element unless the element is expressly recited using the phrase “step for”.

TABLE 1
Summary of Parameters Obtained From
Photopigment Bleaching Calculations

| | | |
|---|---|---|
| Bleaching % | Stimulus Flash [μW] | # of Photons (×10¹¹) |

| | | | |
|---|---|---|---|
| 66% | 45 | μW | 37.7181 |
| 33% | 16.5 | μW | 13.8300 |
| 17% | 7.7 | μW | 6.4540 |
| 8% | 3.45 | μW | 2.8917 |
| 4% | 1.7 | μW | 1.4249 |

1. A method for velocity based optoretinography (ORG), the method comprising:
(a) providing an optical coherence tomography (OCT) system;
(b) applying an optical stimulus to an eye of a subject;
(c) assessing tissue velocity of outer segment tips and inner segment/outer segment junctions of the eye in response to said stimulus with optoretinography (ORG); and
(d) correlating stimulus-evoked tissue velocities with a healthy or diseased state.

2. The method of claim 1, wherein said assessing tissue velocity comprises:
(a) obtaining a 2-dimensional profile of retinal tissues from B-scans of the subject at multiple time points;
(b) assembling the B-scans into a complex M-scan having amplitude and phase that manifest motion of a tissue section; and
(c) assessing tissue velocities from the M-scan.

3. The method of claim 1, wherein said assessing tissue velocity comprises:
(a) serially acquiring a plurality of B-scans from the OCT system;
(b) dividing the B-scans into temporally overlapping blocks; and
(c) performing registration, bulk motion estimation, phase unwrapping, and slope calculation to yield corresponding tissue velocities.

4. A method for velocity based optoretinography (ORG), the method comprising:
(a) obtaining raw spectral data from scanning an eye of a subject;
(b) transforming the raw spectral data into serially-acquired optical coherence tomography (OCT) B-scans;
(c) dividing the B-scans into temporally overlapping blocks; and
(d) performing registration, bulk motion estimation, phase unwrapping, and slope calculation on the blocks to yield corresponding tissue velocities.

5. A method for velocity based optoretinography (ORG), the method comprising:
(a) obtaining optical coherence tomography (OCT) B-scans of an eye of a subject;
(b) selecting groups of sequential B-scans using a moving time window;
(c) using a histogram-based bulk-motion correction algorithm to compensate for axial eye movement during the time window, wherein motion is corrected relative to the first B-scan in the series, and wherein after bulk-motion correction, a resulting complex data cube (V) may be described as:
V(x,z,t)=A(x,z,t)e^{jθ(x,z,t)},
where x and z are the lateral and depth coordinates, respectively, and t is time within the window;
(d) unwrapping the phase data cube θ(x,z,t) in the temporal dimension by adding or subtracting 2π to θ(xₚ,z_{q},tᵣ) in order to minimize θ(xₚ,z_{q},tᵣ)−θ(xₚ,z_{q},tᵣ₋₁)|, where tᵣ and tᵣ₋₁ represent consecutive phase B-scans, wherein this step is performed for each spatial coordinate pair (xₚ,z_{q}) in the volume;
(e) computing a rate of phase change is computed for each coordinate pair by performing a least-squares linear fit with respect to t, giving Δθ/Δt(x,z) in rad/s;
(f) calculating the instantaneous velocity for each spatial location is calculated according to:
$$\frac{Δ z}{Δ t} ( x , z ) = \frac{Δ θ}{Δ t} ( x , z ) \cdot \frac{λ}{4 π n} ,$$
where 1=1060 nm and n=1.38, the nominal refractive index of the eye;
(g) averaging both the B-scan amplitude and
$$( \frac{Δ z}{Δ t} ( x , z ) )$$
in the lateral dimension, giving instantaneous, depth-dependent measures of backscattering and velocity
$$( \frac{Δ z}{Δ t} ( x , z ) ) ,$$
respectively,
(h) shifting the time window by one B-scan period at a time, and constructing a time series of depth profiles, separately for reflectivity and velocity;
(i) visualizing the depth profiles in time-depth coordinates, as M-scans; and
(j) assessing tissue velocities from the M-scans.

6. The method of claim 1, further comprising outputting one or more visualizations of the tissue velocities for correlating stimulus-evoked tissue velocities with a healthy or diseased state.

7. The method of claim 1, further comprising outputting one or more numerical summaries of the tissue velocities for correlating stimulus-evoked tissue velocities with a healthy or diseased state.

8. The method of claim 1, wherein assessment of tissue velocity further comprises measuring the size of retinal substructures.

9. The method of claim 1, wherein the tissues assessed comprise at least one of cone outer segment tips (COST), rod outer segment tips (ROST), and inner segment/outer segment junctions (IS/OS).

10. An apparatus for velocity based optoretinography (ORG), the apparatus comprising:
(a) an optical coherence tomography (OCT) system configured to apply an optical stimulus to an eye of a subject; and
(b) a signal processing unit comprising a processor and a non-transitory memory storing instructions executable by the processor to perform steps comprising:
(i) assessing tissue velocity of outer segment tips and inner segment/outer segment junctions of the eye in response to said stimulus with optoretinography (ORG); and
(ii) correlating stimulus-evoked tissue velocities with a healthy or diseased state.

11. The apparatus of claim 10, wherein said assessing tissue velocity further comprises:
(a) obtaining a 2-dimensional profile of retinal tissues from B-scans of the subject at multiple time points;
(b) assembling the B-scans into a complex M-scan having amplitude and phase that manifest motion of a tissue section; and
(c) assessing tissue velocities from the M-scan.

12. The apparatus of claim 10, wherein said assessing tissue velocity further comprises:
(a) serially acquiring a plurality of B-scans from the OCT system;
(b) dividing the B-scans into temporally overlapping blocks; and
(c) performing registration, bulk motion estimation, phase unwrapping, and slope calculation to yield corresponding tissue velocities.

13. An apparatus for velocity based optoretinography (ORG), the apparatus comprising:
(a) an optical coherence tomography (OCT) system configured to apply an optical stimulus to an eye of a subject, obtain raw spectral data from scanning the eye, and transforming the raw spectral data into serially-acquired optical coherence tomography (OCT) B-scans; and
(b) a signal processing unit comprising a processor and a non-transitory memory storing instructions executable by the processor to perform steps comprising:
(i) dividing the B-scans into temporally overlapping blocks; and
(ii) performing registration, bulk motion estimation, phase unwrapping, and slope calculation on the blocks to yield corresponding velocities.

14. An apparatus for velocity based optoretinography (ORG), the apparatus comprising:
(a) an optical coherence tomography (OCT) system configured to obtain B-scans of the eye;
(b) a signal processing unit comprising a processor and a non-transitory memory storing instructions executable by the processor to perform steps comprising:
(i) selecting groups of sequential B-scans using a moving time window;
(ii) using a histogram-based bulk-motion correction algorithm to compensate for axial eye movement during the time window, wherein motion is corrected relative to the first B-scan in the series, and wherein after bulk-motion correction, a resulting complex data cube (V) may be described as:
V(x,z,t)=A(x,z,t)e^{jθ(x,z,t)},
where x and z are the lateral and depth coordinates, respectively, and t is time within the window;
(iii) unwrapping the phase data cube θ(x,z,t) in the temporal dimension by adding or subtracting 2π to θ(xₚ,z_{q},tᵣ) in order to minimize |θ(xₚ,z_{q},tᵣ)−θ(xₚ,z_{q},tᵣ₋₁)|, where t, and tᵣ₋₁ represent consecutive phase B-scans, wherein this step is performed for each spatial coordinate pair (xₚ,z_{q}) in the volume;
(iv) computing a rate of phase change is computed for each coordinate pair by performing a least-squares linear fit with respect to t, giving Δθ/Δt(x,z) in rad/s;
(v) calculating the instantaneous velocity for each spatial location is calculated according to:
$$\frac{Δ z}{Δ t} ( x , z ) = \frac{Δ θ}{Δ t} ( x , z ) \cdot \frac{λ}{4 π n} ,$$
where λ=1060 nm and n=1.38, the nominal refractive index of the eye;
(vi) averaging both the B-scan amplitude and
$$( \frac{Δ z}{Δ t} ( x , z ) )$$
in the lateral dimension, giving instantaneous, depth-dependent measures of backscattering and velocity
$$( \frac{Δ z}{Δ t} ( z ) ) ,$$
respectively,
(vii) shifting the time window by one B-scan period at a time, and constructing a time series of depth profiles, separately for reflectivity and velocity;
(viii) visualizing the depth profiles in time-depth coordinates, as M-scans; and
(ix) assessing tissue velocities from the M-scans.

15. The apparatus of claim 10, wherein the apparatus provides one or more visualizations of the tissue velocities for correlating stimulus-evoked tissue velocities with a healthy or diseased state.

16. The apparatus of claim 10, wherein the apparatus provides one or more numerical summaries of the tissue velocities for correlating stimulus-evoked tissue velocities with a healthy or diseased state.

17. The apparatus of claim 10, wherein assessment of tissue velocity further comprises measuring the size of retinal substructures.

18. The apparatus of claim 10, wherein the tissues assessed comprise at least one of cone outer segment tips (COST), rod outer segment tips (ROST), and inner segment/outer segment junctions (IS/OS).
